(19) United States
(12) Patent Application Publication
(10) Pub. No.: US 2003/0215509 A1
(43) Pub. Date: Nov. 20, 2003

(54) Coated sustained release tablets of a hygroscopic compound for once-a-day therapy

(75) Inventors: Ashwin Bhujanga Rao, Baroda (IN); Ziauddin Zafir Tyebji, Nagpur (IN); Kamlesh Mohanlal Dudhara, Vadodara (IN)

Correspondence Address: ARMSTRONG,WESTERMAN & HATTORI, LLP, 1725 K STREET, NW, SUITE 1000, WASHINGTON, DC, 20006, US

(73) Assignee: SUN PHARMACEUTICAL INDUSTRIES LIMITED, Mumbai (IN)

(21) Appl. No.: 10/437,042

(22) Filed: May 14, 2003

(30) Foreign Application Priority Data
May 15, 2002 (IN) 440/MUM/2002

(51) Int. Cl. A61K 9/22; A61K 31/19

(52) U.S. Cl. 424/468; 514/557

(57) ABSTRACT

The present invention provides coated sustained release tablets of a hygroscopic compound for once-a-day therapy, said tablets having a moderate weight and volume and comprising—

(a) a core comprising the hygroscopic compound and pharmaceutically acceptable excipients,

(b) a first coating layer comprising a polymer selected from the group consisting of water-insoluble polymers, pH dependent polymers or a mixture thereof, and

(c) a second coating layer comprising a water-insoluble polymer.

### FIELD OF THE INVENTION

The present invention relates to coated sustained release tablets of a hygroscopic compound for once-a-day therapy. Particularly, it relates to coated sustained release tablets of sodium valproate for once-a-day therapy.

### BACKGROUND OF THE INVENTION

Valproic acid and its derivatives, such as sodium valproate, calcium valproate, divlaproex sodium, valpromide, are used widely in the treatment of epilepsy. The effective concentration of the drug in the blood is generally in the range of 50 to 100 μg/ml. Various derivatives of valproic acid such as sodium valproate, calcium valproate, divalproex sodium, valpromide, and combinations thereof are used in the treatment of epilepsy. All these derivatives of valproic acid eventually get converted to valproic acid or the valproate ion, in the gastrointestinal tract.

Sodium valproate is a commonly used derivative of valproic acid, which has a short half-life, and has to be administered three times a day to maintain effective plasma levels of the drug. Multiple dosing of sodium valproate is avoided by formulating sustained release formulations that are indicated for once-a-day therapy in the treatment of epilepsy. However, sodium valproate is water-soluble and highly hygroscopic, and cannot be formulated using conventional sustained release techniques. Also, the treatment of epilepsy requires a large daily dose of sodium valproate reaching 1200 mg, thereby further complicating the formulation procedures. Hence, conventional sustained release tablets of sodium valproate require the use of a large amount of release retarders and excipients, thereby increasing the weight of the tablet.

The prior art includes a number of references on sustained release formulations of valproic acid and its derivatives. U.S. Pat. No. 4,913,906 claims a controlled release oral dosage form comprising a homogenous admixture of 10 to 80 weight percent of valproic acid or its derivatives, and a physiologically acceptable polymer, the dosage form providing prolonged serum level of valproic acid. However, the dosage form is not coated and would be susceptible to the adverse effects of moisture, as a result of the hygroscopic nature of sodium valproate. Also, the sustained release dosage form obtained by the process of the patent is relatively large and difficult to swallow.

U.S. Pat. No. 5,185,159 discloses an orally administrable controlled release tablet pharmaceutical composition comprising a ratio of about one mole valproic acid and about two moles of sodium valproate, and a carrier for controlled release, wherein the tablet is produced without the addition of binder or granulating solvent. The tablet is further coated with a single layer of enteric coating. The core of the tablet is not separated from the enteric coat by any water-insoluble isolating layer. For a hygroscopic active ingredient this would necessitate the use of organic solvents for coating. The use of organic solvents is hazardous from safety and environmental aspects. Thus, the invention is useful only for non-hygroscopic mixtures of valproic acid and sodium valproate, such as divalproex sodium. Also, the tablet weighs 680 mg for 333 mg of sodium valproate and 145 mg of valproic acid, which corresponds to a quite voluminous tablet that some patients find difficult to swallow.

U.S. Pat. No. 5,019,398 discloses a tablet providing sustained release of divalproex sodium for more than 8 hours, wherein the divalproex sodium is present in a matrix comprising hydroxypropyl methylcellulose (8000 to 12000 mPas viscosity) and hydrated silica. The matrix is further coated with a varnish composed of two layers, the first of which is essentially formed of hydroxypropyl methylcellulose (viscosity between 5 and 20 mPas), which is a water-soluble polymer, and the external layer is based on film-forming polyacrylates or polymethacrylates, such as pH-dependent Eudragit® E100 [Poly (butyl methacrylate, (2-dimethyl aminoethyl) methacrylate, methyl methacrylate) 1:2:1] and water-insoluble Eudragit® NE 30D [Poly (ethyl acrylate, methyl methacrylate) 2:1]. The invention is directed to sustained release tablets of divalproex sodium, which is a non-hygroscopic complex formed between one mole of valproic acid and one mole of sodium valproate. Nevertheless, the non-hygroscopic derivative was admixed with an absorbing excipient such as hydrated silica in the core.

U.S. Pat. No. 5,049,586 discloses moisture-stable tablets comprising valproic acid, fillers such as clays or alkaline earth metal oxides, and other pharmaceutically acceptable excipients such as binders, disintegrants and lubricants. There is no coating on the tablets. A tablet containing 500 mg valproic acid weighs approximately 800 mg, when prepared by the process of the patent, as a large amount of clays have to be used to protect the formulation from absorbing moisture. Such large tablets are difficult to swallow.

U.S. Pat. No. 5,169,642 claims a sustained release dosage form comprising granules of divalproex sodium, valproic acid or amides or esters or salts thereof, coated with a sustained release coating composition comprising ethyl cellulose or a methacrylic methylester, a plasticiser and a detackifying agent, the granules being present in a slow release matrix comprising at least one polymeric viscosity agent. The system does not provide two coating layers that provide moisture-stable sodium valproate sustained release tablets.

U.S. Pat. No. 5,980,943 discloses a sustained release dosage form for antiepileptic therapy wherein sodium valproate is coated with a poly(alkylene) oxide or carboxymethyl cellulose polymer, followed by microencapsulation of the coated particles with an aqueous solution of hydroxypropyl methylcellulose. These microencapsulated particles of sodium valproate are then granulated, lubricated and compressed to obtain a core, which is covered with a semipermeable coat having an orifice for the release of the drug. The patent involves complex procedures, and also uses organic solvents for coating. The use of organic solvents is highly hazardous and is best avoided.

### OBJECT OF THE INVENTION

It is an object of the present invention to provide coated sustained release tablets of sodium valproate for once-a-day therapy.

It is a further object of the invention to provide coated sustained release tablets of sodium valproate for once-a-day therapy, such that the tablets have a moderate weight and volume.

It is also a further object of the present invention to provide moisture-stable sustained release tablets of sodium valproate.

### SUMMARY OF THE INVENTION

The present invention provides coated sustained release tablets of a hygroscopic compound for once-a-day therapy, said tablets having a moderate weight and volume and comprising—

(a) a core comprising the hygroscopic compound and pharmaceutically acceptable excipients,

(b) a first coating layer comprising a polymer selected from the group consisting of water-insoluble polymers, pH dependent polymers or a mixture thereof, and

(c) a second coating layer comprising a water-insoluble polymer.

The present invention provides coated sustained release tablets of sodium valproate that are moisture-stable, have a moderate weight and volume and are therefore, easy to swallow. The dual coatings used in the present invention provide moisture stability to the tablets and at the same time provide a sustained release of sodium valproate, such that the tablet is suitable for once-a-day therapy.

### DETAILED DESCRIPTION OF THE INVENTION

The present invention provides coated sustained release tablets of a hygroscopic compound for once-a-day therapy, said tablets having a moderate weight and volume and comprising—

(a) a core comprising the hygroscopic compound and pharmaceutically acceptable excipients,

(b) a first coating layer comprising a polymer selected from the group consisting of water-insoluble polymers, pH dependent polymers or a mixture thereof, and

(c) a second coating layer comprising a water-insoluble polymer.

The hygroscopic compound is any pharmaceutical therapeutically active ingredient having a tendency to pick up water from the environment by any mechanism such as absorption, adsorption and the like. Examples of hygroscopic compounds include sodium valproate, cyanocobalamin, diethylcarbamazine citrate, isosorbide, diclofenac sodium, betahistine mesylate, carpronium chloride, tolazoline hydrochloride, reserpilic acid dimethylaminoethyl ester dihydrochloride, carbachol, choline theophyllinate, choline salicylate, citicoline, hexamethonium bromide, chlorophyllin sodium-copper salt, panthenol, dexamethazone phosphate disodium salt, plant extracts, procainamide hydrochloride, L-proline, potassium chloride and calcium chloride.

Preferably the hygroscopic compound of the present invention is sodium valproate. The core comprising sodium valproate and pharmaceutically acceptable excipients is surrounded by a dual coating, wherein the first coating layer comprises a polymer selected from the group consisting of water-insoluble polymers, pH dependent polymers or a mixture thereof, and the second coating layer comprises a water-insoluble polymer. The second coating may further comprise a polymer selected from the group consisting of a water-soluble polymer, a pH dependent polymer, or mixtures thereof. Such a dual coating provides moisture-stable sodium valproate tablets, and at the same time avoids the use of moisture absorbing excipients in the core, thus limiting the weight and size of the tablets. The dual coating also provides sustained release of sodium valproate from the core for a period of more than 8 hours, the tablets therefore providing once-a-day therapy of sodium valproate.

The core of the present invention comprises sodium valproate in an amount ranging from about 80% to about 95% by weight of the tablet. In preferred embodiments, the amount of sodium valproate used is 500 mg.

The core of the present invention may include various pharmaceutically acceptable excipients, for example disintegrants such as starch, cellulose derivatives, gums, crosslinked polymers and the like; binders such as starch, gelatin, sugars, cellulose derivatives, polyvinyl pyrrolidone and the like; lubricants such as calcium silicate, talc, magnesium stearate, colloidal silicon dioxide and mixtures thereof.

Examples of binders used commonly include starch, gelatin, sugars like sucrose, glucose, dextrose, molasses and lactose; acacia, sodium alginate, cellulose derivatives like methyl cellulose, ethyl cellulose, carboxymethyl cellulose and the like; polymers such as polyvinyl pyrrolidone, Veegum, polyethylene glycol, waxes and the like.

In a preferred embodiment of the present invention, polyvinyl pyrrolidone is used as the binder. Vinyl pyrrolidone polymers or polyvinyl pyrrolidone (PVP), also referred to as Povidone, are synthetic polymers consisting essentially of linear 1-vinyl-2-pyrrolidinone groups, the degree of polymerization of which results in polymers of various molecular weights, the molecular weight ranging between 2500 and 3,000,000 Daltons. PVP is commercially available as Kollidon® (BASF), Plasdone® and Peristone® (General Aniline). PVP is classified into different grades on the basis of its viscosity in aqueous solution. Different grades of PVP available are PVP K-12, PVP K-15, PVP K-17, PVP K-25, PVP K-30, PVP K-60, PVP K-90 and PVP K-120. The K-value referred to in the above nomenclature is calculated from the viscosity of the PVP in aqueous solution, relative to that of water. The preferred vinyl pyrrolidone polymer for use as a binder is PVP K-30, having an approximate molecular weight of 50,000 Daltons. It may be used in an amount ranging from about 0.5% to about 5% by weight of the core, more preferably from about 1% to about 2% by weight of the core.

Microcrystalline cellulose (MCC) is used in a preferred embodiment of the present invention as a wicking agent. It is made up of a chain of about 250 glucose molecules in the form of a microcrystal, consisting primarily of crystallite aggregates obtained by removing amorphous regions of a pure cellulose source material by hydrolytic degradation using mineral acid. MCC has an average molecular weight of about 36,000 Daltons and is available in various grades, which differ in bulk density, particle size and moisture content. It is commercially available as Vivapur®, Avicel®, Vivacel®, Emcocel®, Fibrocel® and Tabulose®. It is used in an amount ranging from about 1% to about 5% by weight of the core, more preferably from about 3% to about 4% by weight of the core.

Examples of lubricants that may be used in the coated sustained release tablets of the present invention include talc, magnesium stearate, calcium stearate, aluminium stearate, stearic acid, calcium silicate, hydrogenated vegetable oils, colloidal silicon dioxide, polyethylene glycol, cellulose derivatives such as carboxyalkyl cellulose and its alkali salts, or mixtures thereof. In a preferred embodiment of the present invention, a mixture of calcium silicate, magnesium stearate and talc is used as the lubricant, in an amount ranging from about 0.5% to about 5% by weight of the core.

The core of the coated sustained release tablets of the present invention is coated with a first coating layer comprising a water-insoluble polymer, a pH dependent polymer or a mixture thereof. The water-insoluble polymers and the pH-dependent polymers that may be used include water-insoluble cellulose derivatives such as ethyl cellulose, and various grades of methacrylic acid copolymers, the different grades being dependent on the methacrylic acid content and the viscosity of the methacrylic acid polymers. In preferred embodiments, the core is surrounded by a

first coat comprising a water-insoluble polymer, such as a cationic polymer based on dimethylaminoethyl methacrylate and other neutral methacrylic acid esters. Preferably, poly(butyl methacrylate, (2-dimethyl aminoethyl) methacrylate, methyl methacrylate) (1:2:1), available worldwide under the brandname Eudragit E 100, is used as the water-insoluble polymer. Eudragit E 100 provides a film that is soluble in gastric fluid below pH 5, and is not a release rate controlling polymer. It is used in an amount ranging from about 0.1% to about 5% by weight of the core.

The core of the coated sustained release tablets of the present invention is further surrounded by a second coating layer comprising a water-insoluble polymer, such that the coat provides a controlled release of the sodium valproate from the core. The second coating layer may further comprise a polymer selected from a group consisting of a water-soluble polymer, a pH dependent polymer, or mixtures thereof. The water-insoluble polymers that may be used in the second coating layer include cellulose derivatives such as ethyl cellulose, cellulose esters, various grades of methacrylic acid copolymers, or mixtures thereof In preferred embodiments, the second coating layer comprises a mixture of a cellulose ester and a methacrylic acid copolymer. Preferably, the cellulose ester used is cellulose acetate. Various grades of cellulose acetate, which differ in their acetyl content and degree of polymerization, are commercially available, and may be used in the present invention. It is used in an amount ranging from about 0.5% to about 5% by weight of the tablet. In preferred embodiments, the cellulose acetate used is cellulose acetate 398-10 NF grade, having an acetyl content of 39.8% and a number average molecular weight of 40,000. The methacrylic acid copolymer used in the second coating layer is preferably an ammoniomethacrylate copolymer, such as poly(ethyl acrylate, methyl methacrylate, trimethylammonioethyl methacrylate chloride) (1:2:0.2), commercially available under the brandname Eudragit RL 100. Eudragit RL 100 provides a film that provides a sustained release of the sodium valproate from the core. It is preferably used in an amount ranging from about 1% to about 5% by weight of the tablet.

The first and second coating layers may further contain a plasticiser such as glycerol, propylene glycol, polyethylene glycol, sorbitol, diethyl phthalate, mineral oil, triacetin, triethyl citrate, and the like. The plasticisers are generally used in an amount ranging from about 0.01% to about 1% by weight of the tablet.

The coated sustained release tablets of the present invention may be obtained by a process wherein the hygroscopic compound, such as sodium valproate, is mixed with the pharmaceutically acceptable excipients and granulated using a binder and a granulating solvent. The granules thus obtained are dried, lubricated with a lubricant or a mixture of lubricants, and compressed to obtain a core. The core is then coated with a solution of the first coating layer. The tablets are dried and further coated with a solution of the second coating layer using conventional methods known to a person skilled in the art. The tablets are finally dried to obtain the coated sustained release tablets of the present invention.

The following example does not limit the scope of the present invention and is used as an illustration.

### EXAMPLE 1

The coated sustained release tablets of sodium valproate are obtained as given in Table 1 below.

TABLE 1

| | | |
|---|---|---|
| Ingredients | Quantity (mg/tablet) | Quantity (% w/w) |

Core

| | | |
|---|---|---|
| Sodium valproate | 500.0 | 82.06 |
| Polyvinyl pyrrolidone (PVP K- | 6.95 | 1.14 |
| 30) | | |
| Microcrystalline cellulose | 24.15 | 3.96 |
| Calcium silicate | 15.625 | 2.56 |
| Magnesium stearate | 6.25 | 1.03 |
| Talc | 9.525 | 1.56 |
| First coating | | |
| Eudragit E100 | 4.02 | 0.66 |
| Polyethylene glycol (PEG 6000) | 0.80 | 0.13 |
| Talc | 3.21 | 0.53 |
| Titanium dioxide | 3.21 | 0.53 |
| Second coating | | |
| Cellulose acetate 398-10NF | 16.83 | 2.76 |
| Eudragit RL100 | 7.21 | 1.18 |
| Triethyl citrate | 3.60 | 0.59 |
| Talc | 5.29 | 0.87 |
| Titanium dioxide | 2.64 | 0.43 |

Sodium valproate, polyvinyl pyrrolidone and microcrystalline cellulose is passed through American Standard for Testing an Materials (ASTM) #60 sieve and mixed to obtain a uniform blend. This dry blend is then granulated using water as the granulating agent. The wet mass obtained upon granulation passed through ASTM #20 sieve and the granules thus obtained are dried at a temperature of 60° C. The dried granules are again passed through ASTM #20 sieve. Calcium silicate, magnesium stearate and talc are passed through ASTM#60 sieve and mixed with the dry granules. This lubricated mass is then compressed into cores using 18 mm×9 mm capsule-shaped punches. The first coating layer solution is obtained by dissolving Eudragit E100 in a mixture of acetone and isopropanol, with an aqueous solution of PEG 6000, and a dispersion of talc and titanium dioxide in isopropanol added to it. The coating solution is sprayed on the cores by conventional methods known to a person skilled in the art and the cores are dried. The second coating layer solution is obtained by dissolving cellulose acetate and Eudragit RL100 in acetone, with triethyl citrate and a dispersion of talc and titanium dioxide in isopropanol added to it. It is sprayed onto the cores using conventional methods known to a person skilled in the art. The cores with the dual coating are then dried to obtain the coated sustained release tablets of the present invention.

The coated sustained release tablets of sodium valproate thus obtained are subjected to dissolution testing using United States Pharmacopoeia apparatus Type I, using 900 ml of distilled water as the dissolution medium, at 100 rpm. The results of the dissolution test are recorded in Table 2 below.

TABLE 2

| Time (hours) | % drug released (±SD) |
|---|---|
| 2 | 38.81 ± 11.16 |
| 4 | 67.37 ± 13.01 |
| 8 | 86.75 ± 8.73 |
| 12 | 95.45 ± 4.75 |

While the invention has been described with reference to specific embodiments, this was done for purposes of illustration only and should not be considered to limit the spirit or the scope of the invention.

1. Coated sustained release tablets of a hygroscopic compound for once-a-day therapy, said tablets having a moderate weight and volume and comprising—
(a) a core comprising the hygroscopic compound and pharmaceutically acceptable excipients,
(b) a first coating layer comprising a polymer selected from the group consisting of water-insoluble polymers, pH dependent polymers or a mixture thereof, and
(c) a second coating layer comprising a water-insoluble polymer.

2. Coated sustained release tablets as claimed in claim 1, wherein the hygroscopic compound is sodium valproate.

3. Coated sustained release tablets as claimed in claim 1, wherein the second coating layer further comprises a polymer selected from the group consisting of water-soluble polymer, a pH dependent polymer, or mixtures thereof.

4. Coated sustained release tablets as claimed in claim 1, wherein the pharmaceutically acceptable excipients include polyvinyl pyrrolidone and cellulose derivatives.

5. Coated sustained release tablets as claimed in claim 4, wherein the polyvinyl pyrrolidone is used in an amount ranging from about 0.5% to about 5% by weight of the core.

6. Coated sustained release tablets as claimed in claim 4, wherein the cellulose derivative used is microcrystalline cellulose.

7. Coated sustained release tablets as claimed in claim 6, wherein the microcrystalline cellulose is used in an amount ranging from about 1% to about 5% by weight of the core.

8. Coated sustained release tablets as claimed in claim 1, wherein the first coating layer comprises a water-insoluble polymer.

9. Coated sustained release tablets as claimed in claim 8, wherein the water-insoluble polymer is a methacrylic acid copolymer.

10. Coated sustained release tablets as claimed in claim 9, wherein the methacrylic acid copolymer is used in an amount ranging from about 0.1% to about 5% by weight of the core.

11. Coated sustained release tablets as claimed in claim 9, wherein the methacrylic acid copolymer is poly(butyl methacrylate, (2-dimethyl aminoethyl) methacrylate, methyl methacrylate).

12. Coated sustained release tablets as claimed in claim 1, wherein the second layer comprises a mixture of a cellulose ester and a methacrylic acid copolymer.

13. Coated sustained release tablets as claimed in claim 12, wherein the cellulose ester is present in an amount ranging from about 0.5% to about 5% by weight of the tablet, and the methacrylic acid copolymer is present in an amount ranging from about 1% to about 5% by weight of the tablet.

14. Coated sustained release tablets as claimed in claim 13, wherein the cellulose ester used is cellulose acetate and the methacrylic acid copolymer used is poly(ethyl acrylate, methyl methacrylate, trimethylammonioethyl methacrylate chloride).

15. Coated sustained release tablets as claimed in claim 1, wherein the coating layers further comprise a plasticiser.
